(19) United States
(12) Patent Application Publication
(10) Pub. No.: US 2024/0032817 A1
(43) Pub. Date: Feb. 1, 2024

(54) Hand-held medical diagnostic device and methods of generating rapid medical diagnostics using artificial intelligence

(71) Applicant: Simpli-Fi Automation, Inc., Davenport, FL (US)

(72) Inventor: Christopher Campbell, Davenport, FL (US)

(21) Appl. No.: 18/316,057

(22) Filed: May 11, 2023

Related U.S. Application Data
(60) Provisional application No. 63/496,885, filed on Apr. 18, 2023.
(60) Provisional application No. 63/340,625, filed on May 11, 2022.

(51) Int. Cl. A61B 5/08; G01N 33/98; A61B 5/0205; A61B 5/097; G01N 33/497; A61B 5/00; B82Y 15/00

(52) U.S. Cl. CPC A61B 5/082; G01N 33/98; A61B 5/02055; A61B 5/097; G01N 33/497; A61B 5/742; A61B 5/7267; A61B 5/7271; A61B 5/7221; B82Y 15/00; A61B 2562/0285; A61B 2562/029; A61B 2562/125; A61B 2562/166; A61B 2562/0271; A61B 2562/046; A61B 5/021

(57) ABSTRACT

An athletic performance analysis device. The device includes a chemically functionalized single wall carbon nanotube sensor array for the discrimination and quantification of volatile organic compounds (VOC's) or chemical biomarkers in human body fluids, a plurality of biometric sensors to gather data on physical characteristics of body function, and artificial intelligence machine learning models to process the collected sensor data for the purpose of rendering a comprehensive assessment of the functionality and performance readiness of an athletes body. The system further comprises a remote user interface, a communication interface arranged to send the collected breath biomarker and biometric data, a storage module arranged to store the received measurements, and a display arranged to display information to the user based on the compiled machine learning model analysis.

### CROSS-REFERENCE TO RELATED APPLICATIONS

The present application claims the benefit of U.S. Provisional Application No. 63/340,625 filed May 11, 2022, and U.S. Provisional Application No. 63/496,885, filed Apr. 18, 2023, which applications are hereby incorporated in their entirety by reference.

### FIELD OF THE INVENTION

The invention relates to the field of wearable technology and medical devices for sports medicine and athletic training.

### BACKGROUND

Sports medicine has a long and rich history dating back to ancient times when athletes in Greece and Rome would seek medical attention to recover from injuries sustained during athletic competitions. However, it wasn't until the early 20th century that the field of sports medicine began to take shape as a distinct medical specialty.

Sports medicine is a field of medicine that focuses on the diagnosis, treatment, and prevention of sports-related injuries and conditions. Athletic readiness assessment is a key area within sports medicine that involves evaluating an athlete's physical and mental condition to determine their ability to perform at a high level and minimize the risk of injury.

In recent years, the field of sports medicine has seen significant innovation in the area of athletic readiness assessment. One major area of innovation has been the use of wearable technology to track an athlete's physical condition in real-time. Wearable devices such as heart rate monitors, motion sensors, and GPS trackers can provide coaches and trainers with valuable information about an athlete's fitness level, exertion, and performance.

Another important innovation in the field of athletic readiness assessment has been the use of advanced imaging techniques such as X-rays, MRIs, and CT scans to diagnose and monitor sports-related injuries. These imaging techniques allow medical professionals to see inside the body and detect injuries that may not be visible to the naked eye.

In addition to these technological innovations, there has been a growing emphasis on the importance of nutrition and mental health in athletic readiness assessment. Athletes are increasingly turning to nutritionists and mental health professionals to optimize their diets and mental well-being, respectively, in order to perform at their best.

Recent innovations in the use of SWCNT breath biomarkers represent another exciting innovation in the field of athletic readiness assessment. These chemical biomarkers can provide valuable insights into an athlete's metabolic and hormonal activity, which can in turn be used to monitor and optimize their performance and recovery. The challenge is that to date, the testing and analytical equipment for this analysis is bulky, expensive, and requires a trained professional to use.

What is needed is a hand-held device that can gather and analyze chemical breath biomarker data while an athlete is in the field of play or training. When combined with biometric sensor data, this chemical breath analysis data can provide comprehensive, real-time analysis of an athlete's athletic performance and recovery readiness. This device would constitute a substantial advancement in the field.

### SUMMARY

The above-mentioned needs are, in large measure, addressed by embodiments of the present disclosure. Embodiments of the invention disclosed herein include systems, devices, and methods for the real-time chemical breath and biometric sensor analysis of an athlete's physical performance readiness and recovery levels. Embodiments of the present disclosure include a hand-held fitness tracking device that uses artificial intelligence to combine breath biomarker chemical analysis with electronic biometric sensor data to provide a comprehensive assessment of an athlete's physical performance readiness and recovery trends.

In certain embodiments of the disclosed invention, a hand-held breath testing apparatus, contains an air inlet that is connected to a breath sampling chamber containing a functionalized single wall carbon nanotube sensor array (SWCNT), a type of chemical sensor that is used to detect and identify specific molecules in a gas or liquid sample. The air inlet is used to receive exhaled air from a human user's lungs and pass the sample air through the chemiresistor testing chamber and out an exhaust opening.

In preferred embodiments, the SWCNT array can include multiple individual SWCNT sensors that are functionalized with specific chemical groups to selectively interact with the target molecules. When a breath sample is introduced to the sensor array, the target molecules selectively bind to the functionalized SWCNTs, causing a change in the electrical resistance of the SWCNTs. This change in resistance is then measured and recorded by the sensor array, providing a unique electrical signal pattern that is specific to the target molecule.

According to an aspect of the disclosure, the chemiresistor is constructed as a two-terminal device, with each individual SWCNT sensor acting as an independent sensing element. The SWCNTs are deposited onto a substrate surface in a specific pattern to create an array of sensors. The electrical resistance of the SWCNTs in the sensor array is measured using various techniques, including current-voltage (IV) measurements or impedance spectroscopy. These techniques enable the measurement of the complex impedance pattern of the sensor array, which can be used to extract the real and imaginary components of the electrical signal.

In a preferred embodiment, the chemiresistor can be constructed in a 16-32 sensor array made with nine different sensing materials to achieve selective discrimination. As an example, Carboxylic SWCNTs, sulfonated SWCNTs, hydroxyl-functionalized SWCNTs and polyaniline are used to obtain room temperature detection of methane. In the same embodiment purified SWCNTs, polypyrene, graphene, SWCNT functionalized with polyethylene glycol (PEG) and Pd doped SWCNT can be used as additional sensing materials for the sensor array. At least two polymers can be used to achieve a good degree of selectivity as the sensing mechanism for polymer-based sensors is different from that for CNT-based sensors.

In yet another embodiment, the sensor array is fabricated using printed circuit board (PCB) as the substrate. The array consisted of 32 interdigitated electrodes, each with a finger gap size of 120 μm. All sensing materials are deposited and pipetted across the interdigitated electrode pattern of the sensor platform. Each material is deposited in three channels. The evaporation of the solvent leaves a network of nanotubes on the electrodes to bridge the interdigitated fingers. The base resistance of the sensors can be measured to be in the range of 500 ohm to 15 Kohm. The thickness of the film in each case can be adjusted to get the desired base resistance.

Preferred embodiments of the present invention include methods of using machine learning algorithms to analyze the resistance patterns of the SWCNT sensor array to identify and quantify a wide range of VOCs and chemical biomarkers including Acetone, Ethane, Pentane, Isoprene, Nitric Oxide, Carbon Dioxide, Lactic Acid, and more. These chemicals are related directly to the health and functionality of key physiological systems related to athletic performance, so this data is used to provide valuable real-time insights into an athlete's physical condition and readiness for competition.

In certain embodiments of the invention, the hand-held device contains a plurality of biometric sensors such as heart rate, blood pressure, bio-electrical impedance analysis, ECG, airflow, temperature, digital stethoscope, and humidity that are used to validate the chemical analysis and provide a substantially higher rate of accuracy than breath biomarker analysis alone.

In one embodiment, the system combines breath biomarker data and biometric sensor readings. An example is used wherein the SWCNT sensor can read elevated levels of Carbon Dioxide in the breath, which indicates increased physical exertion levels and oxygen saturation, causing the system to check the plurality of biometric sensors with expectation to see elevated heart rate and blood oxygen readings from the integrated pulse oximeter. This correlation enables the system to render a more accurate analysis of the athlete's physical condition.

According to an aspect of the disclosure, the device contains a micro-processor that is used to analyze the data provided by the SWCNT sensor. The micro-processor is then connected to a Wi-Fi, Bluetooth, or LoraWan communications module for output to a mobile communication device associated with the subject.

This disclosure includes methods for using artificial intelligence (AI) machine learning models that combine and process the collected chemical breath and biometric sensor data to render an accurate analysis profile of the internal bio-chemical function of a user.

In some embodiments of the invention, the system can further comprise a remote user interface, a communication interface arranged to send the collected data, a data storage module arranged to store the received measurements, and a display arranged to display information to the user based on the compiled AI model analysis.

In a preferred embodiment, certain algorithms can be made operable to create custom nutritional supplement recommendations based upon the outcomes of the breath test. The algorithm will list the component gases that reach a threshold of intervention. As an example, if the test shows a low level of acetone, which indicate a deficiency in the breaking down of fat, the algorithm will be used to select the appropriate amount of Biothin Probiotic (250 mg) Nutritional supplements that counteract the effect of the metabolic imbalance as demonstrated by elevated acetone.

In further embodiments, the system can be integrated with third party health and fitness devices to gather additional sensor data from fitness watches, smart weight scales, and wearable health and fitness technologies. The device software uses API integrations to query connected fitness data and then compile the data to create a comprehensive user interface dashboard of all the collected devices to provided training insights. The invention represents a significant advancement in the field of sports medicine and athletic readiness assessment, providing coaches and trainers with a powerful tool for optimizing athlete performance and reducing the risk of injury.

### BRIEF DESCRIPTION OF THE DRAWINGS

FIG. 1 shows a handheld fitness tracking device in accordance with an example of the present invention.

FIGS. 2A-2F show perspective views of a handheld fitness tracking device in accordance with examples of the present invention.

FIG. 3 shows a sensor chip in accordance with an example of the present invention.

FIGS. 4A-4B show NO₂ testing data in accordance with an example of the present invention.

FIGS. 5A-5B show Cl₂ testing data in accordance with an example of the present invention.

FIGS. 6A-6B show interference data in accordance with an example of the present invention.

FIG. 7 shows humidity data in accordance with an example of the present invention.

FIG. 8 shows time-dependent function data in accordance with an example of the present invention.

FIGS. 9A-9E show AEPV amplitudes for sensors in accordance with an example of the present invention.

FIGS. 10-13 show a method of chemical breath and biometric sensor analysis in accordance with an example of the present invention.

FIGS. 14A-14D show testing data for sensor materials in accordance with an example of the present invention.

FIG. 15 shows testing data for target gases in accordance with an example of the present invention.

FIGS. 16A-16B show principle component analysis of gases in accordance with an example of the present invention.

FIG. 17 shows sensor testing data in accordance with an example of the present invention.

### DESCRIPTION

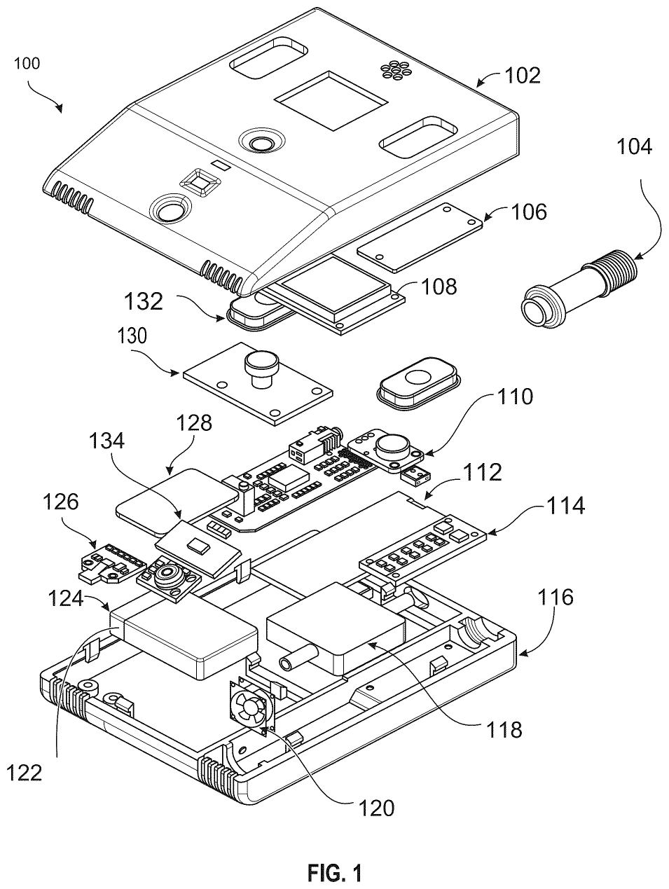

FIG. 1 illustratively shows a handheld fitness tracking device 100. As shown, handheld fitness tracking device 100 may include electronic modules in the form of EKG sensor pads 132, a 5-direction joystick controller 130, a ECG sensor module 128, a pulse oximeter 134, an environmental sensor 126, a battery 124, a temperature sensor 122, a top casing 102, a UV LED module 106, a LCD screen 108, a puffer tube 104, a MEMS audio microphone/digital stethoscope 110, a control processor 112, a SWCNT gas sensor array 114, a lower casing 116, an airflow sensor 118, and an exhaust fan 120.

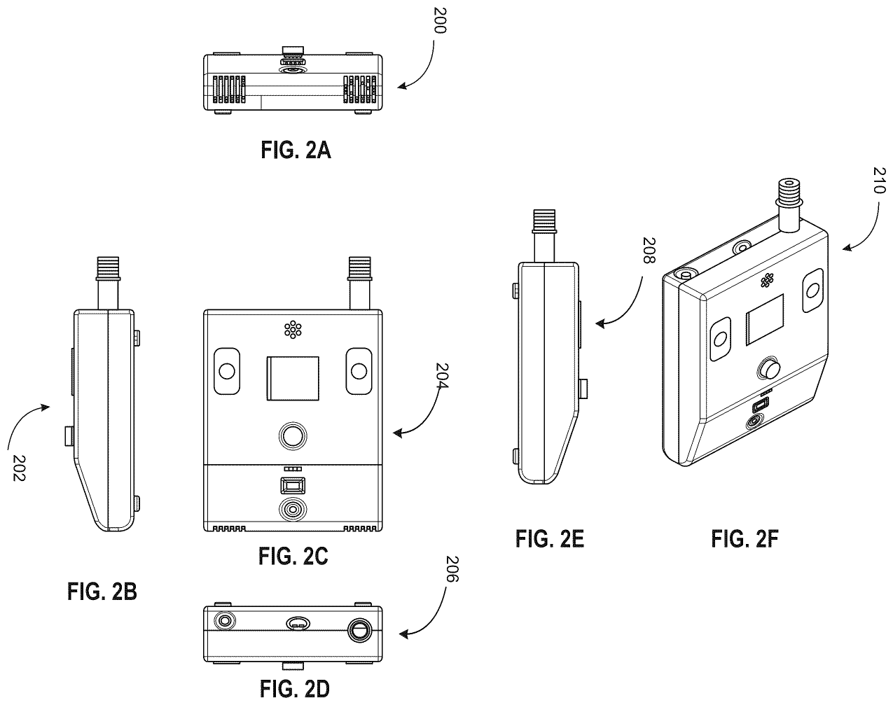

FIGS. 2A-2E illustratively show perspective views of a handheld fitness tracking device in accordance with examples of the present invention. Specifically, FIG. 2A shows a bottom/exhaust view of a handheld fitness tracking device 200 in accordance with an example of the present invention. FIG. 2B illustratively shows a right-side view of a handheld fitness tracking device 202 in accordance with an example of the present invention. FIG. 2C shows a front view of a handheld fitness tracking device 204 in accordance with an example of the present invention. FIG. 2D illustratively shows a top/inlet view of a handheld fitness tracking device 206 in accordance with an example of the present invention. FIG. 2E is a left side view of a handheld fitness tracking device 208 in accordance with an example of the present invention. Finally, FIG. 2F is a graphic rendering of a handheld fitness tracking device 210 in accordance with an exemplary example of the present invention.

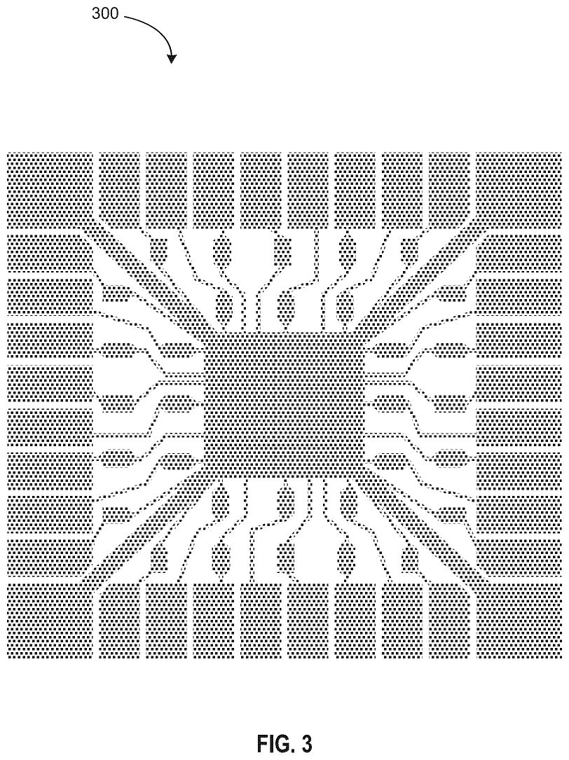

FIG. 3 shows a sensor chip in accordance with an example of the present invention. A sensor chip 300 includes 32 sensor elements on a 1×1 cm² area divided into four banks of eight sensors. These banks are denoted as A, B, C, and D for convenience, starting from the bottom and moving counterclockwise. An individual sensor element is 500×300 lm² in size. The microfabrication approach described below enables reduction of the chip size and increasing the number of sensor elements. Though current fabrication is on 4⁹⁹ wafers, scaling up to larger wafers is straightforward. Each chip is mounted on a ceramic carrier and includes a heater, a temperature sensor, and a humidity sensor.

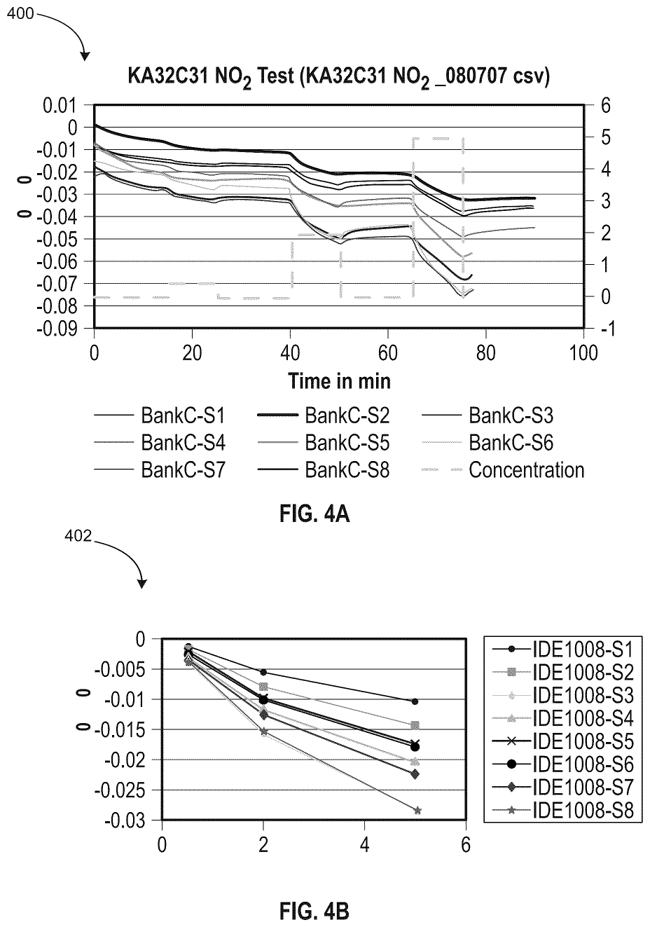

FIGS. 4A and 4B illustratively show NO₂ testing data in accordance with an example of the present invention. Beginning with FIG. 4A, testing 400 shows a response of one bank (bank C) of 8 single-walled carbon nanotube (SWCNT) sensors for NO2 exposure at 0.5, 2, and 5 ppm in air at room temperature. The concentration (y-axis on the right side) is shown as step functions. In FIG. 4B, testing 402 shows a response versus NO2 concentration for the eight sensors. The response is represented as normalized resistance (R-R)/Rₒ, where Rₒ is the baseline resistance and R is the instantaneous resistance. The sensor chips were first exposed to NO2 only at 0.5, 2, and 5 ppm in air. The exposure sequence and exposure times are as shown in FIG. 4. FIG. 4 shows the response curves for the eight sensors in bank C (finger gap of 12 lm and finger width of 10 lm). The response plotted here is the normalized resistance (R-Ro)/Ro where Rₒ is the baseline resistance and R is resistance at time t.

Also plotted in FIG. 4 is the normalized resistance versus concentration calibration curves for the eight sensors. The first NO2 exposure occurs at t 5 15 min with 0.5 ppm. Even during the first 15 min with no gas exposure, the resistance continues to change; during this time known as the “conditioning period” in the sensor literature, the sensor is on with a voltage and the resistance is expected to change. It is well known that the baseline is likely to drift over time in all types of chemiresistor-based sensors for a variety of reasons. FIG. 4 shows that the sensor is sensitive down to 0.5 ppm and the behavior is similar to our earlier NO2 work¹¹ wherein an extrapolation of a calibration curve like the one in FIG. 4 showed a detection limit of 4 ppb.

The behavior of four out of the eight sensors in FIG. 4 is very close to each other with others showing some variation. As mentioned earlier, the manual process of ink jetting may result in variations of the amount of dispersion dispensed and thus the resulting nanotube density from sensor-to-sensor. This is not really a flaw since the response curve of each sensor is stored as its unique fingerprint for that analyte for pattern recognition purposes later. What is more important is chip-to-chip variation on the same wafer (which is affected by the same factor as above plus many others) as it would be ideal not to have to train every single chip and instead, load the training data from one chip onto all others.

Analysis of all the four banks of the sensors indicates a stronger response for A with the largest finger gap of 50 lm. The variation between sensor elements in each bank does not show any marked dependence on finger gaps. In any case, a systematic study and optimization are needed.

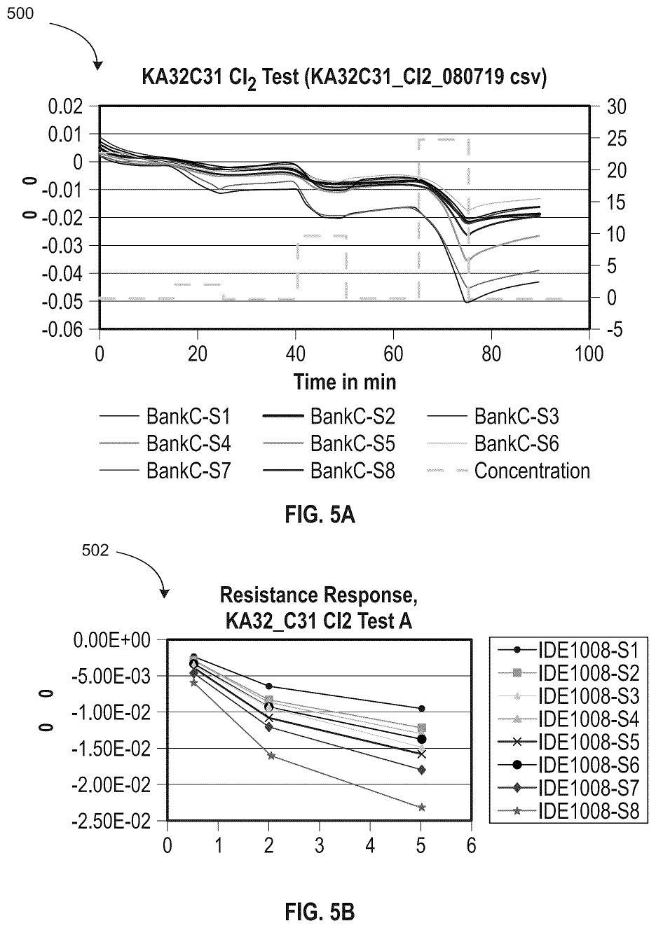

FIGS. 5A and 5B illustratively show Cl₂ testing data in accordance with an example of the present invention. Beginning with FIG. 5A, testing 500 shows a response of one bank (bank C) of 8 SWCNT sensors for Cl2 at 6, 10, and 15 ppm in air at room temperature. FIG. 5B testing 502 shows response versus chlorine concentration calibration curves. Next, after a thorough purging, the sensor chips were exposed to Cl2 at 6, 10, and 15 ppm in air. The response curves and calibration curves for the same bank C sensor elements are depicted in FIG. 5, which show clear responses to chlorine exposure. In general, the response signal for chlorine can be enhanced if the SWCNTs are coated with chloro-sulfonated polyethylene as depicted in Refs. 14 and 15. Next, the sensor chips were tested for their response to NO2 in a high concentration of chlorine background gas. The nitrogen dioxide concentration was fixed at 0.5 ppm, whereas the chlorine level was varied at 6, 10, and 15 ppm sequentially in separate tests.

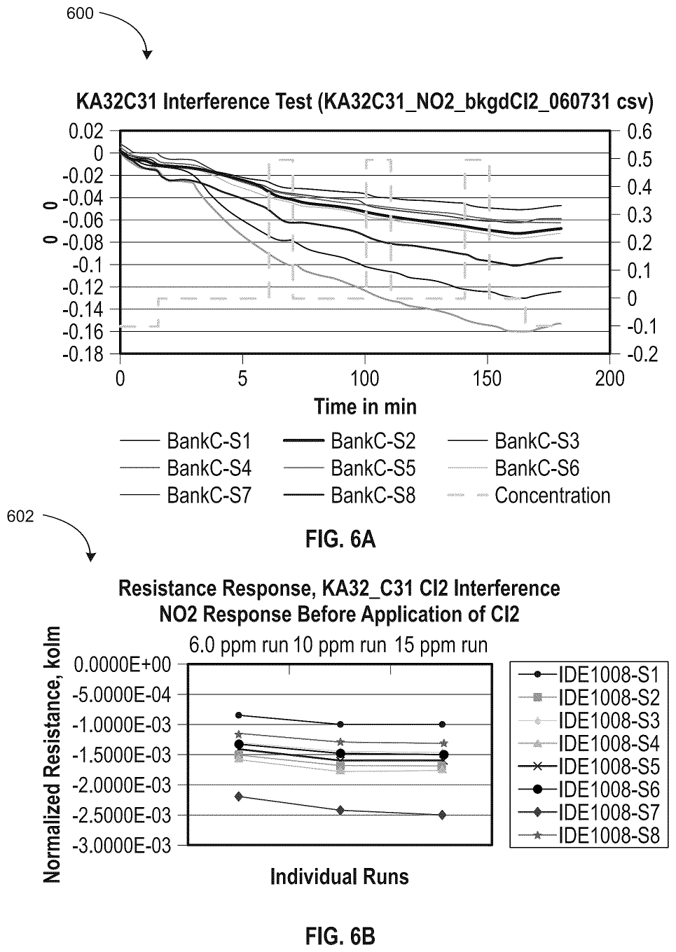

FIGS. 6A and 6B illustratively show interference testing data in accordance with an example of the present invention. More specifically, the sensing of NO2 in an interfering background of Cl2. Beginning with FIG. 6A, testing 600 illustratively shows the response of one bank (bank C) of 8 SWCNT sensors for 0.5 ppm of NO2 three times on top of 10 ppm Cl2 in air at room temperature. FIG. 6B testing 602 shows sensor responses of 8 SWCNT sensors to 0.5 ppm NO2 on top of 6, 10, and 15 ppm of Cl2 in air at room temperature. FIG. 6 shows distinct sensor responses to 0.5-ppm NO2 with a 10-ppm-Cl2 background in air after each NO2 injection. The response peaks represent the overlay of the nitrogen dioxide over the existing flow of chlorine.

In each case, the NO2 application clearly changes the trend of the existing line. The consistent and nearly constant sensor responses of 0.5-ppm NO2 on top of 6, 10, and 15 ppm in FIG. 6b indicate that the chlorine gas does not interfere with NO2 detection. Although the kinetics of any possible reactions are not known and the overall relationships are hard to define, the results here present evidence that the chips can detect the presence of NO2 in a background of Cl2 up to 30× more concentration and, up to this point, with no evidence of sensor saturation or failure to return to baseline.

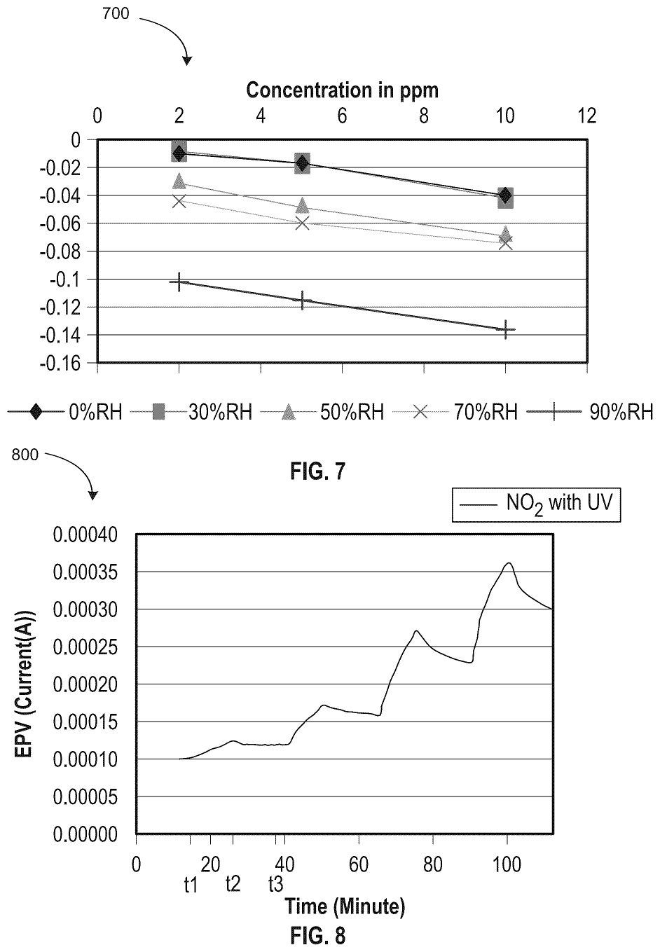

FIG. 7 illustratively shows humidity data in accordance with an example of the present invention. Specifically, testing 700 the effect of humidity on response to NO2 exposure. Relative humidity was varied between 0 and 90%. Humidity is a common interfering factor in chemical sensor operation. Several mechanisms have been suggested to understand the humidity effect including electron donation to CNTs, bonding of hydrogen with the oxygen defect sites in CNTs, and direct H2O adsorption onto CNTs leading to charge trapping sites. The situation of cross sensitivity with the analyte of interest and background can be even more complex.

It is important to establish the impact of this effect on sensor performance, and for this reason, the NO2 study was repeated at various relative humidity levels. FIG. 7 shows the normalized sensor response (R-Ro)/Ro as a function of NO2 concentration for four relative humidity levels between 0 and 90%. The dependence on humidity does not appear to be linear, perhaps indicating the need for a few more data points if interpolation was to be used during signal processing to correct for the prevailing humidity.

FIG. 8 illustratively shows time-dependent function data in accordance with an example of the present invention. Specifically, testing 800 graphically illustrates a time dependent function that is measured for NO2 and used to implement the analysis. The function EPV(t;meas), representing an electrical parameter value, is measured and recorded for a selected electrical parameter value, such as electrical current, voltage difference, resistance, impedance, conductance or capacitance associated with an electrical circuit that is partly or wholly io constructed using nanostructures (“NSs”) that may be, but need not be, carbon-based.

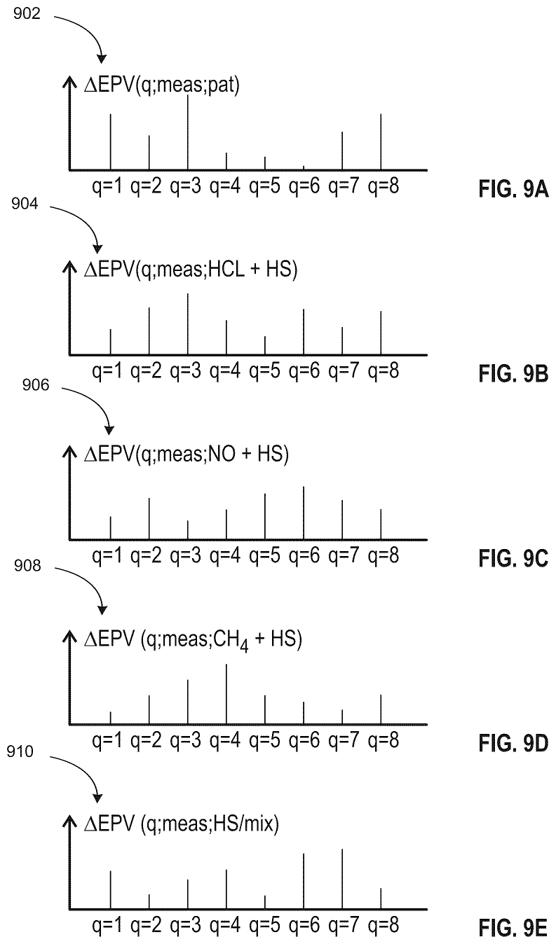

FIGS. 9A-9E illustratively show graphs of AEPV amplitudes for Q distinct sensors, numbered q=1, . . . , Q (Q2; Q=8 here) from a sample gas measured from the user. Specifically, FIG. 9A shows data 902 of a sample gas measured from the user. FIG. 9B shows data 904 of a first reference sample having a known concentration of HC1 plus HS gas. FIG. 9C shows data 906 of a second reference having a known concentration of NO plus HS gas. FIG. 9D shows data 908 of a third reference sample having a known concentration of CH₄ plus HS gas. Finally, FIG. 9E shows data 910 of a gas from one or a mixture of persons (not including the patient) that are known to have none of the diseases associated with the specified compound.

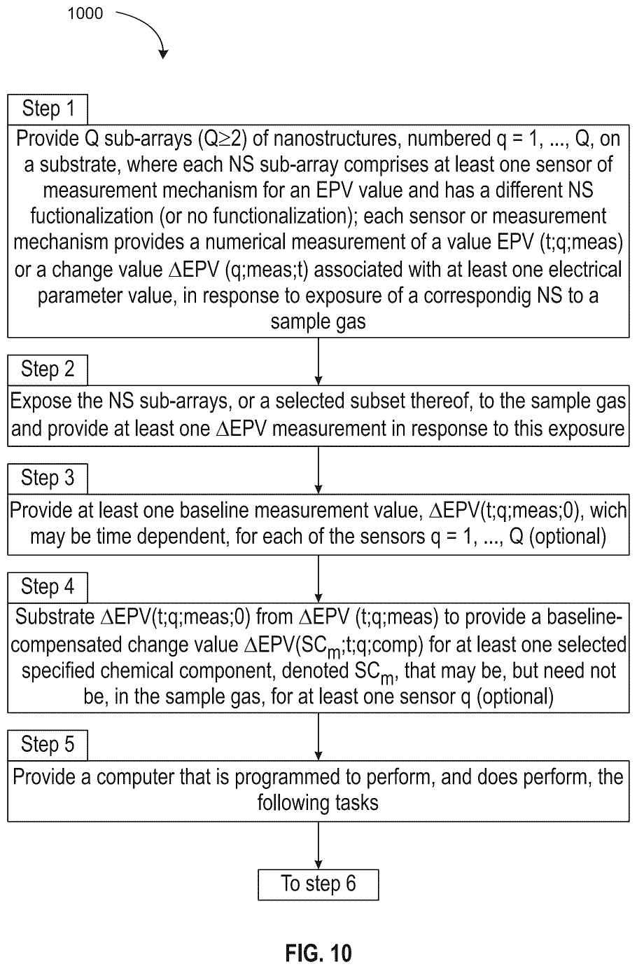

FIG. 10 shows a flow chart of a procedure for practicing an embodiment of the invention. Flow chart 1000 illustratively begins with step 1 where Q sub-arrays of nanostructure (NS) sub-arrays (Q>1) are provided on a substrate, where each NS sub-array comprises at least one sensor or measurement mechanism for an EPV value and has a different NS functionalization (or no functionalization). Each sensor comprises at least first and second interdigitated electrodes, with each electrode being connected at a first electrode end to at least one of (i) a controllable voltage source and (ii) a controllable current source and the first and second ends of an electrode being connected to each other in an electrical path that includes one or more of the functionalized (or non-functionalized) nanostructures. Each sensor or measurement mechanism provides a numerical measurement associated with a change in an electrical parameter value AEPV (t;q;meas), representing a change in at least one of electrical voltage difference, electrical current, impedance, resistance, conductance, inductance or another relevant EPV value, in response to exposure of a corresponding NS to a sample gas.

In step 2, the NS sub-arrays, or a selected subset thereof, are exposed to the sample gas and at least one AEPV measurement is provided in response to this exposure. In step 3, at least one baseline measurement AEPV, denoted AEPV (t;q;meas;0), which may be time dependent, is provided for at least one measurement time value t. (optional). In optional step 4, a baseline measurement AEPV (t;q;meas;0) is subtracted from a AEPV measurement value to provide a baseline-compensated AEPV value AEPV(t;q;meas;base), 50 which may depend, but need not depend, upon a time value.

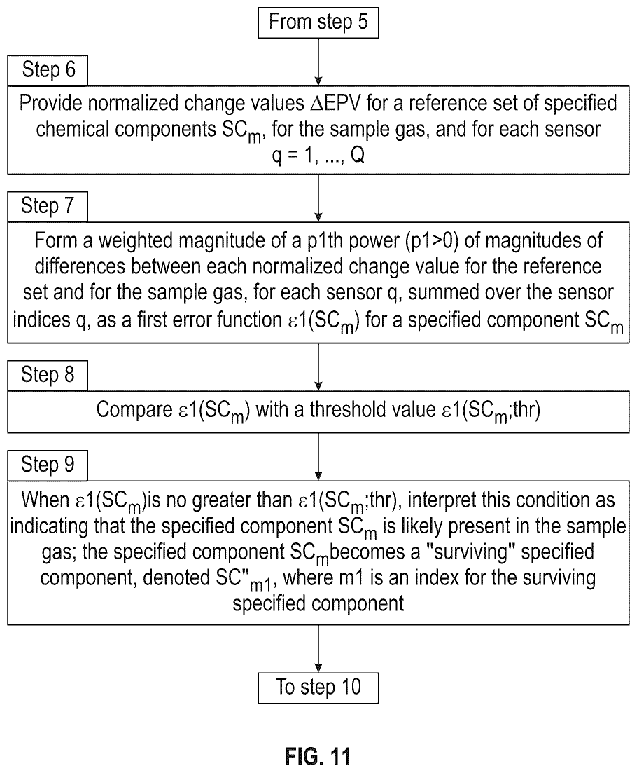

In step 5, a computer is provided that is programmed to perform, and does perform, the following tasks illustratively shown in FIG. 11. Beginning with step 6, normalized change values AEPV for a reference set of specified chemical components SC,,, and for the sample gas are formed, as set forth in Eqs. In step 7, for each specified component SCm (a candidate for inclusion in the sample gas) and for each sensor q, a first error function s 1(SCₘ) is formed as a pth power of weighted magnitudes of differences between the normalized change values for the reference set and for the sample gas, and these weighted differences are summed over the sensors, q=1, . . . , Q, as set forth in Eq. (5), where the weighting parameters u_{q} are non-negative and the sum of the weighting parameters over the Q sensors is equal to a positive constant (e.g., 1).

In step 8, the numerical value of the first error function s 1 (SCm) is compared with a first threshold value s 1 (SCm;thr), which may vary, but need not vary, with the specified component. In step 9, when s1(SCm₁) is no greater than s1(SCm;thr), the system interprets this condition as indicating that the specified component SCm is likely present in the sample gas. A first subset of specified components SC,,, that satisfy the condition in step 48 become a surviving subset of specified components, ISC″m₁1, where m1 is an index for this first subset. When s 1 (SC″m) is greater than s 1 (SCm;thr), the system interprets this condition as indicating that the specified component SCm, likely (i) is not present in the sample gas or (ii) is present in the sample gas with a negligible concentration K(SCm;pat. The presence of this second subset of specified components in the sample gas is optionally ignored.

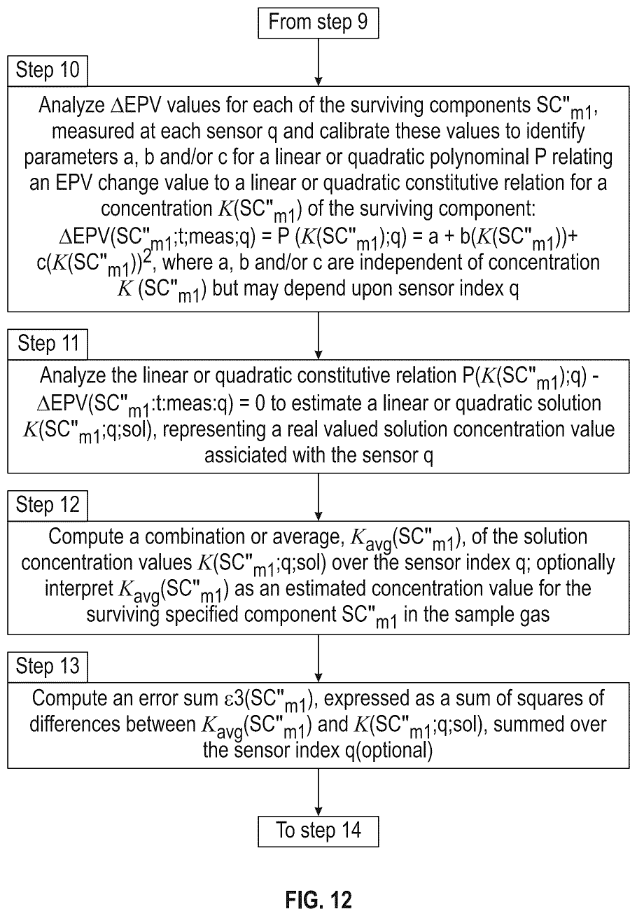

Turning to FIG. 12, in step 10, each of the set of surviving specified components SC″ml measured at each sensor q, is analyzed or calibrated as in Eqs. (9)-(26), using known concentrations (indexed by r) of the surviving components with their measured change values AEPV(K(SC″m1q;r)), to identify parameters a, b and/or c, for which a linear polynomial approximation:

ΔEPV(SC″ₘ;q;approx)=a+bκ(SC″ₘ),

or, a quadratic polynomial approximation:

ΔEPV(κ(SC″ₘ₁;q;approx))ₐ+bκ(SC″ₘ)+cκ(SC″ₘ₁)²,

provides a best linear fit or a best quadratic fit, respectively, for a fixed surviving specified component SC″m1, and a fixed sensor q, for the reference set of concentration values (r=1, . . . , R). The parameters a, b and c are independent of concentration values K(SC″ₘ;r) but may depend upon the choice of surviving specified compound SC″,,,,,₁; and/or upon the choice of sensor q. Turning to step 11, a linear or quadratic constitutive relation is analyzed. A linear constitutive relation,

a′+b′κ(SC″ₘ₁;q)−ΔEPV(SC″ₘ₁;q)=0,

or a constitutive relation,

a′+b′κ(SC″ₘ;q)+c′κ(SC″ₘ;q)²−ΔEPV(SC″ₘ₁;q)=0,

to represent a real valued solution concentration value associated with a sensor q. In step 12, a combination or average, Kav_{g}(SC″m₁) is computed, representing an average, of the solution concentration values k(SC″m;q;sol), over the index q, for example as set forth in Eq. (29) or 30), is formed. The corresponding value Kavg (SC″m1) is optionally interpreted as an estimated concentration value for the surviving specified component SC″m1 in the sample gas. In step 13, a sum of a square of differences between Kav_{g}(SC″m₁) and each of the sum of solution concentration values K(SC″;q;sol) is formed, and an error sum s3(SC″m1), over the sensor index q is computed.

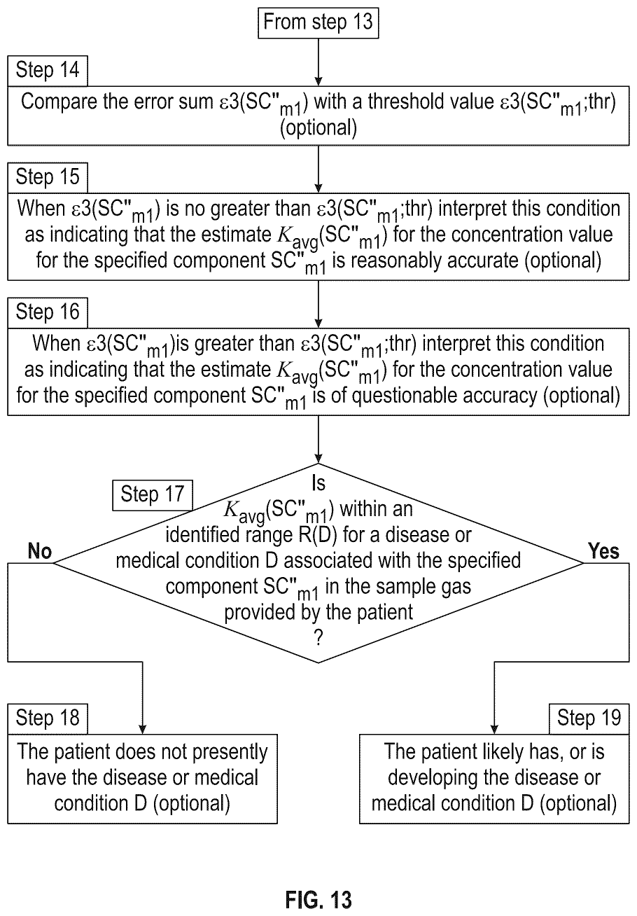

Turning to FIG. 13, optionally, in step 14, the error sum s3(SC″m₁) is compared with a threshold value s3(SC″m1;thr). Optionally, in step 15, when s3(SC″m₁) is no greater than s3(SC″m1;thr), the system interprets this condition as indicating that the estimate Kavg(SC″M1) for the concentration value for the surviving specified component SC″m1 in the sample gas is reasonably accurate. Optionally, in step 16, when s3(SC″m1) is greater than s3(SC″m₁;thr), the system interprets this condition as indicating that the estimate Kavg(SC″m1) for the concentration value for the surviving specified component SC″m₁ in the sample gas has questionable accuracy. In step 17, the system determines whether Kavg(SC″m1) is within an identified range R(D) for a physiological condition D that is associated with presence of the surviving specified component SC″m₁ in the sample gas provided by the user. If yes, optionally, in step 19, the system interprets this condition as indicating that the user is likely to have, or to be developing, the physiological condition in question. If no, optionally, in step 18, the system interprets this condition as indicating that the physiological condition D is not likely present in the user.

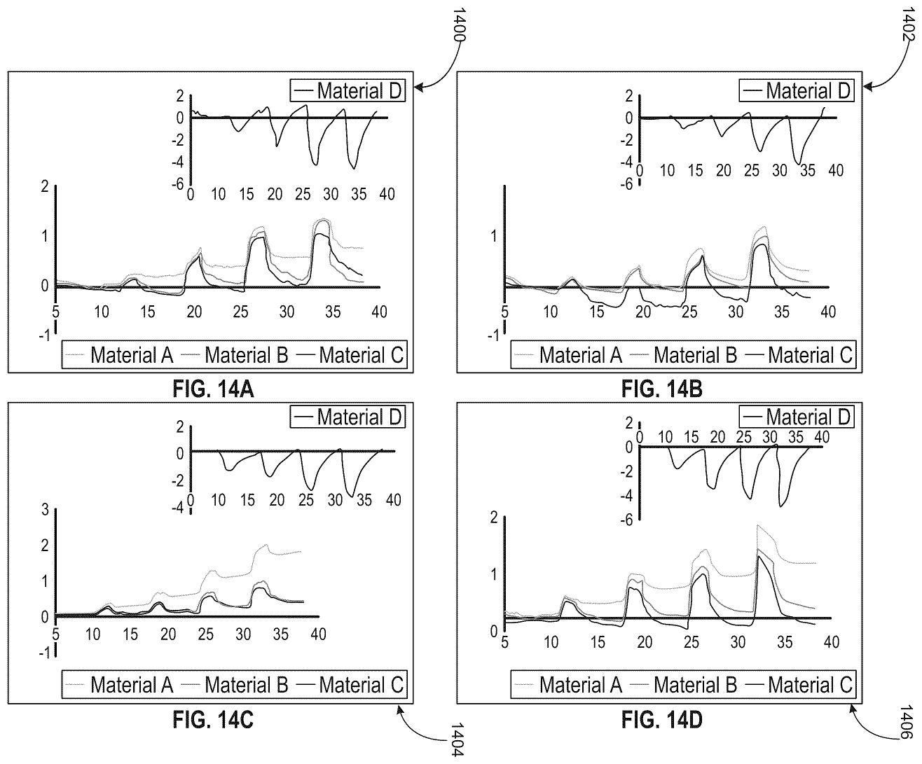

FIGS. 14A-14D illustratively show testing data for sensor materials in accordance with an example of the present invention. Beginning with FIG. 14A, data 1400 illustratively shows sensor responses of various sensor materials to 2, 5, 10, and 25 ppm of CH4. Alternatively, data 1402 in FIG. 14B shows sensor responses of various sensor materials to 2, 7, 17, and 27 ppm CO. Additionally, data 1404 in FIG. 14C shows sensor responses of various sensor materials to 2, 5, 12, and 27 ppm SO2. Finally, data 1406 in FIG. 14D shows sensor responses of various sensor materials to 2, 5, 10, and 30 ppm NH3. In each chart, sensor material A represents carboxylic-SWCNTs, material B represents sulfonated-SWCNTs, material C represents hydroxyl-SWCNTs, and material D polyaniline.

In general, as shown, the sensor chip was exposed to CH4, CO, SO2, and NH3 in the concentration range of 1-30 ppm at the interval shown. The corresponding calibration curves providing the response vs. concentration are given in Figure S4. The sensors were purged with an airflow rate of 400 cm³/min for the first 10 min, as well as before and after any target gas exposures. All the tests were performed at room temperature. FIG. 14 shows the response curves for four of the nine materials to various concentrations of the target gases. The response plotted here is the normalized resistance (R′R0)/R0, where R0 is the baseline resistance before gas exposure and R is the instantaneous resistance at any time t after the gas exposure. It is well known that chemiresistive sensors generally drift with time and therefore, signal processing is done by looking at the relative change in the slope of the response curve when gas exposure occurs.

The conductivity change of the sensors is concentration dependent and it increases or decreases linearly with gas concentration in the range of 1-30 ppm for various gases in a unique manner. Sensor responses to various gases depend on both the chemical nature of the sensing material and the target gas. The electrical resistance of the three SWCNT-based materials in FIG. 14 increased, while it decreased for polyaniline upon exposure to various target gases.

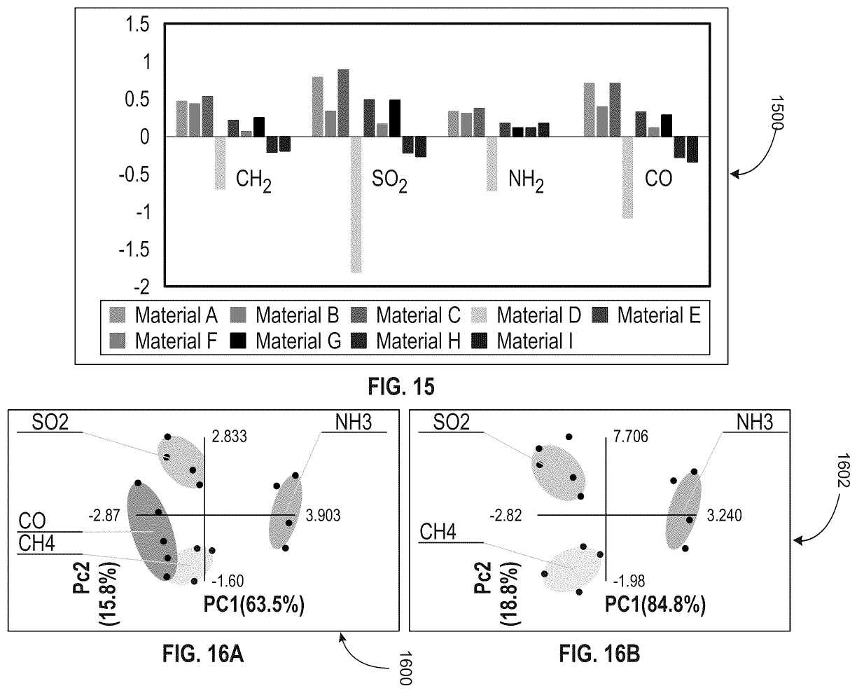

FIG. 15 shows testing data for target gases in accordance with an example of the present invention. More specifically, testing data 1500 shows the normalized responses in a bar chart for all nine materials for 25 ppm each of CH4, CO, SO2, and NH3 exposures. Polyaniline provides a much stronger response than the SWCNT based materials for all gases; however, experience indicates performance degradation over time for polymer-based materials whereas the CNTs are more stable over longer periods. Within the observed response region in FIG. 3, the three SWCNT-based candidates from FIG. 4 show distinct responses for each of the four analytes. Though each material is used in three sensor channels of the sensor array (Table S1), the sensor-to-sensor variation among the three can be 5-10% based on extensive characterization in the lab for various material-gas combinations. This is largely due to the manual drop-casting of the sensing material onto the chip and this variability is expected to go down when automated ink-jetting or an equivalent process is used. Generally, FIG. 15 shows normalized responses in bar chart form for 25 ppm concentrations of target gases Ch4, So2, NH3 and CO. The error bars range from 1.5% to 6%. All three functionalized SWCNTs in Fig G exhibit an increase in resistance upon exposure to CH4, CO, SO2, and NH3 gases while polyaniline shows the opposite behavior. Doped polyaniline is widely used to detect acidic and basic gases and the conductivity of polyaniline depends on both the oxidation state of the main polymer chain and the degree of protonation on imine sites. Any interaction with polyaniline is N-type doped. In some cases, the polymer-target gas interaction can be attributed to hydrogen bonding as well as dipole-dipole interactions.

In another example, carboxylic-SWCNTs treated in HNO3:H2SO4 mixture for 2 h did not show significant response to CH4 (data not shown here). Treatment lasting longer times (˜4 h) yielded much improved response to CH4. It is possible that the longer acid treatment might have introduced a greater number of defects and carboxylic groups, allowing better sensitivity to the target gases. The presence of oxygenated functionalities at the ends of the SWCNTs facilitates electron transfer with target gases. The larger response with oxygenated carbon nanotubes might be the result of introduction of more controlled carboxylic groups, which forms low-energy adsorption sites and facilitates charge transfer at defect sites. Similar results are observed and reported with sulfonated SWCNTs. The change in conductivity resulting from the interaction of certain gases with functionalized-SWCNTs has also been ascribed to the formation of hydrogen bond between the functionalized group and the target gas molecule. Like carboxylic-SWCNTs, sulfonic acid defects and hydroxyl defects also form low energy absorption sites and facilitate charge transfer at defects sites. Sulfonic acid group (SO3F) is even more acidic than the carboxylic group.

FIGS. 16A-B show principle component analysis of gases in accordance with an example of the present invention. More specifically, data 1600 of FIG. 16A shows a principle component analysis of sensor array response to CH4, CO, SO2 and NH3, while data 1602 of FIG. 16B shows a principle component analysis of a sensor array response to CH4, SO2 and NH3. PCA Study: After exposing the nine different materials to various gases, the sensor discrimination ability was studied using principal component analysis, a technique widely used in electronic nose data analysis to discriminate gases/vapors. The analysis expresses the main information in the variables by lowering the number of variables, the so-called principle components. It is an orthogonal projection of data from a higher dimensional space to a lower dimensional one so that the variance of the projected data is maximized. The data matrix was constructed with the rows representing the response of each sensing material to different gasses at two different concentrations (15 and 25 ppm). FIG. 16A shows that each gas cluster displaying a clear distance between then except that the CO cluster interferes with CH4. FIG. 16B shows that CH4 can easily be discriminated from SO2 and NH3. Further discrimination between CH4 and Co would require materials, which behave differently to these gases.

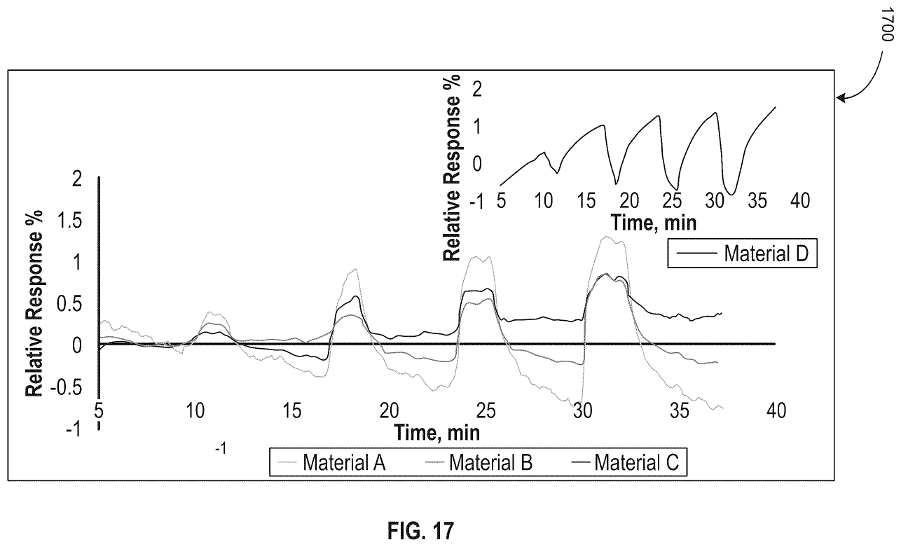

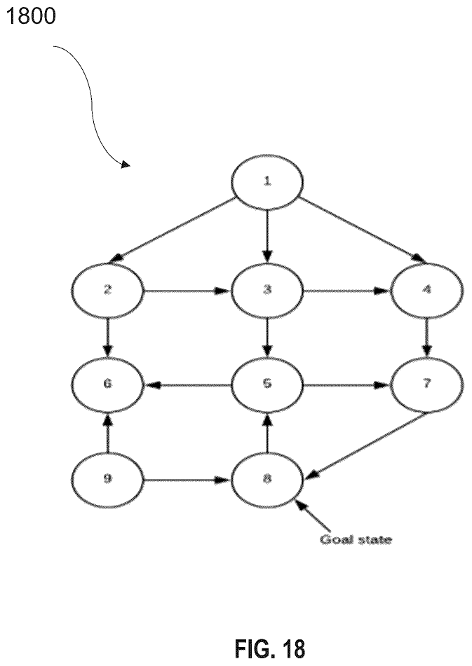

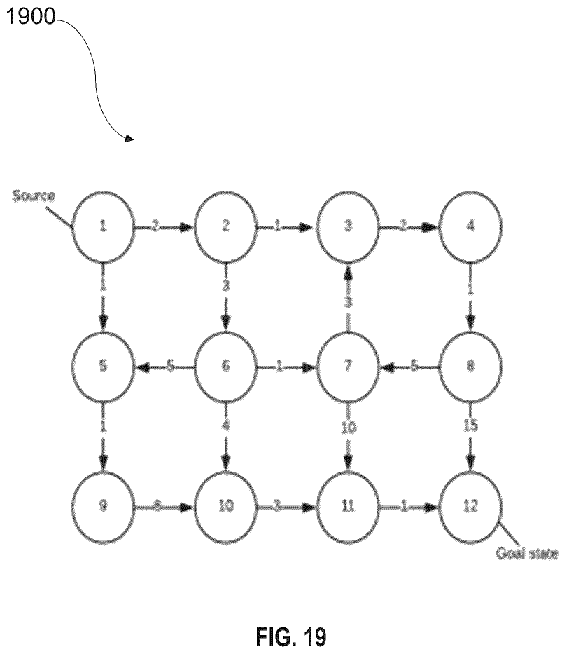

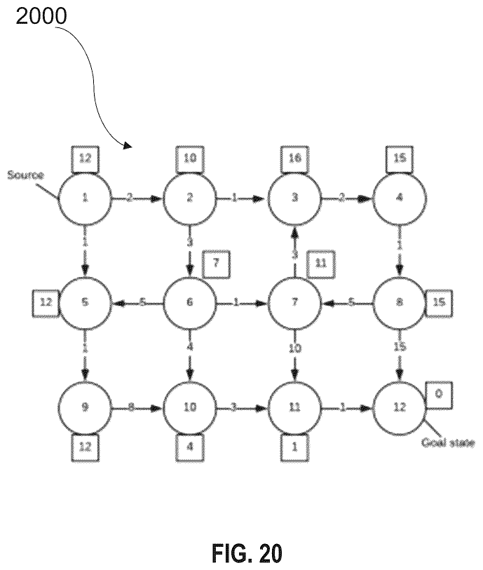

Finally, FIG. 17 shows sensor testing data in accordance with an example of the present invention. Specifically, data 1700 of FIG. 17 shows responses of various sensor materials to 2, 5, 10, and 25 ppm CH4 using a SWCNT sensor module.

The preferred embodiment of the present invention has the overall properties of a hand-held athletic performance and recovery profiling and analysis device. The device utilizes an array of 32 coated or doped Single Wall Carbon Nano Tube chemiresistive sensors to detect Volatile Organic Compounds (VOC), Non-Volatile Compounds (non-VOC), and molecular chemical compounds on the breath of a target subject as they relate to the metabolic function and overall athletic readiness and recovery vales of a target subject. The device also makes operable a plurality of biometric sensors to gather data on the health and activity of a user's internal organs and systems.

In a preferred embodiment, the system processes aggregated data from the SWCNT sensor array, the plurality of integrated biometric sensors, and third-party fitness device application programming interfaces (API's) and uses artificial intelligence machine learning models to create a comprehensive and comparative assessment of a user's athletic performance readiness level. This data includes, but is not limited to; metabolic function, physiological conditions, phycological states, and athletic recovery rates.

In a further embodiment, the SWCNT sensor detects discrete molecular compounds related to metabolic function, athletic performance, muscle recovery, fatigue, and other biometric parameters and then compares those detected particles to a database of know athletic performance biometric biomarkers to determine the athletic readiness and recovery status of an individual user.

In an embodiment, the present invention contains multiple communication methods. The device contains a mini-Bluetooth module, Wi-fi-communication module and a low bandwidth, low power, wide-area network antenna (LORAWAN). This ensures that the device can be used in any environment or even completely stand alone. In this embodiment, the device can communicate with almost any Bluetooth device, IoT network, or Data network.

In another embodiment, the device has an exterior shell with biometric sensors embedded and accessible from the top and face of the device. Those sensors include an EKG sensor, a pulse oximeter, a 3.5 mm EEG electrode sensor input, and a body temperature sensor, a digital stethoscope, a video camera, an environmental gas, and humidity sensor. Most of these sensors are electrically integrated with the micro-processor board using a I2C communication bus. Some of the sensors require analog I/O inputs and are connected to individual analog inputs on the microprocessor.

In the present embodiment, the device contains a gas sampling chamber wherein a disposable air puffer tube or straw is physically inserted. The user expels the air completely out of his/her lungs directly into the puffer tube. This exhausted breath is then passed thru the gas sampling chamber in which a SWCNT sensor array is situated horizontally on the lower section of the chamber nearest to the puffer tub. As the gas sample is passed over the SWCNT sensor, many of the molecules or VOCs in the sample will deposit onto the face of the sensors and cause a reactive change in the electrical resistance of the sensor based upon a chemical reaction with the functionalization layer of the individual SWCNT chemiresistive sensor. Each sensor is chemically functionalized to have a predictable change in electrical parameter values upon contact with one or more analytes.

In vet another embodiment, the sample gas chamber contains a horizontally positioned array of 10 miniature ultraviolet light emitting diodes and a heating element for the purpose of sterilizing the breath sensor chamber. The heating element is placed in the center of the SWCNT array on the lower section of the chamber with the uv LED array situated on the surface of the top section of the testing chamber. Upon completion of a sampling procedure, the UV LED's and heating element are activated inside the chamber and expose the SWCNT sensor face to local heating of the NS sub-array with energy density of the order of 1-100 Joules/cm² for 10-30 sec and irradiation of the NS sub-array with ultraviolet-emitting LEDs (e.g., with wavelengths in a range (e.g., 254-256 nm) for 1-100 seconds.

In a preferred embodiment, the present invention contains in the lower section of the device housing the environmental Sensor, rechargeable battery, microprocessor, memory storage, Micro Bluetooth module, air-flow sensor, SWCNT sensor array, mini EEG sensor module, and heating element. All sensors are hardwired to the microprocessor.

In another preferred embodiment, the present invention contains in a upper section of the device housing a no-contact temperature sensor, pulse oximeter sensor module, 5 direction controller, OLED screen monitor, EKG sensor pads, electronic stethoscope, and mini digital camera. At the top of the housing is the puffer tub insertion hole, EEG 3.5 mm input, and USB type C jack.

In a certain embodiment, the SWCNT sensing material is prepared by selecting, pristine Single-walled carbon nanotubes then first chemically modifying them by acid treatment to obtain carboxylic-SWCNTs and sulfonated-SWCNTs. The SWCNTs are acid refluxed at 120° C. for about 4 hours to allow sufficient time for reaction and achieve a high level of carboxylic functionalization. Carbon nanotubes are also functionalized using potassium hydroxide to obtain hydroxyl (OH) functionalized SWCNTs. These functionalization processes increase the surface activity and enable easier interaction with the target gases.

In a most preferred embodiment, the sensor fabrication is done on a p-type silicon (100) Wafer with a resistivity of 0.006-0.01 cm and an approximate thickness of 500 jtm. Patterned platinum (Pt) lines (200-nm thick) are deposited on top of thermally grown 0.5-jtm SiO2 for the purposes of electrical heating and resistive temperature detection. Then a 1-jtm Si3N4 layer is deposited as an insulating layer, followed by the IDE pattern. This consists of 200-nm Pt on top of 20-nm Ti with varying finger gaps and finger widths. The finger gaps are 50, 25, 12, and 8 jtm for sensors in banks A, B, C, and D, respectively. The finger width is 20 jtm for sensors in bank A and it is 10 jtm in the remaining three banks. These are the only (physical) variations among the sensor elements, and in this work, chemical variations such as dopings or coatings have not been introduced, instead using only pristine SWCNTs. This is adequate because pristine CNTs are known to be sensitive to NO2.

In a further instance of the present embodiment, bulk SWCNTs are used, consisting of semi-conducting and metallic varieties and no attempt was made to separate or eliminate the metallic tubes. After thorough purification, the nanotubes are dispersed in a solvent (dimethyl formamide) and inkjetted across the IDE pattern of the sensor platform (typically, 0.05 jtL). Following the quick evaporation of the solvent a thin film of nanotubes bridges across the electrodes. Manual inkjetting to each sensor element in a sensor array is the current approach. However, doing this across the whole wafer could be impractical for quality control.

In addition, this leads to sensor-to-sensor variation within the same bank of sensors. As of now, an automated delivery system with X-Y movement with appropriate needles for nanoliter drop size is not available. For this reason, an in-situ chemical vapor deposition approach can be developed to deposit the SWCNTs directly on the sensor platform. In-situ growth of CNTs for chemical sensing is a way to scale up the chemical sensor fabrication on a wafer scale. When optimized, this approach can provide uniform deposition of CNTs either as a sensing material or as a supporting matrix for coating and doping other binding materials for sensing purpose.

In yet another embodiment of the present invention, the sensor chips are wire bonded into a carrier through which individual resistances of sensors could be measured; each chip is tested with the multichannel ohmmeter that also comprises the data acquisition system for the gas testing. Current-voltage characteristics are measured (Keithly Instruments 2002/7001, Scottsdale, AZ) for each individual sensor on the chips. Then the chips can be tested for their response to NO2 and Cl2 individually, and the results compared to previous results for these two gases and also with each other to ensure internal consistency in the data. This is followed by a set of tests where the concentration of chlorine was set constant and NO2 was pulsed for 10 min at equal concentrations, with recovery periods in between each trial.

In another instance of the embodiment, the background concentration can vary but it does not pose any complications since the actual sensing involves looking for known fingerprints from the data bank for the analytes in question and identification with the aid of a pattern recognition algorithm. Gas exposure at specified concentrations is done using a computerized multicom-ponent gas blending and diffusion system (Environics 2000; Environics Inc., Tolland, CT). A steady total flow of 400 cc/min can be used during exposure to various gas streams.

In a further embodiment, the functionalized nanotubes are then dispersed in a solvent and sonicated for 2 h. Protonated polyaniline is used as another sensing material in the sensor array to help with discriminating between various gases. Polyaniline base is protonated using strong hydrochloric acid to obtain a polyaniline salt. The green precipitate is filtered, washed with distilled water and dried in a vacuum oven for 3 h at 40° C. These four materials are synthesized specifically to achieve enhanced room temperature response to CH4 as an example.

In yet another embodiment, to enhance selectivity of the sensor array, commercially available materials, such as purified nanotubes, polypyrrole, graphene, PEG-functionalized SWCNTs, and Pd-decorated SWNTs can be used. The choices made here for sensing materials are meant to be representative; additional and/or different materials from a range of possibilities including functionalized graphene, other two-dimensional materials, nanoparticles and metal oxide nanowires can be used to provide wide chemical variations in constructing the multichannel sensor array chip.

In yet another version of an embodiment of the present invention, the SWCNT sensor is fabricated using printed circuit board (PCB) as the substrate. The array can consist of 32 interdigitated electrodes, each with a finger gap size of 120 μm. All sensing materials are deposited and pipetted across the interdigitated electrode pattern of the sensor platform. Each material is deposited in three channels. The evaporation of the solvent leaves a network of nanotubes on the electrodes to bridge the interdigitated fingers. The base resistance of the sensors can be measured to be in the range of 500 ohm to 15 Kohm. The thickness of the film in each case can be adjusted to get the desired base resistance.

In another of the same version of the embodiment, the network is doped with a transition element, such as Pd, Pt, Rh, Ir, Ru, Os and/or Au, and change in an electrical parameter value is again analyzed. SWCNTs yield different signal responses when exposed to different gases and vapors and one must use pattern recognition or intelligent signal processing techniques for the identification of the gas constituent of interest. SWCNTs do not respond to exposure to certain gases and vapors, and in those cases, coating or doping of the nanotubes may elicit a signal.

In the same version of another preferred embodiment, hydroxypropyl cellulose, having a mass of 0.791 gm, was dissolved in a solvent of 25 ml chloroform for coating the nanotubes to detect presence of HCl. In each case, an aliquot of 5 ml polymer solution can be drop-deposited onto the Single-Walled Carbon Nanotube (SWCNT) to coat the corresponding SWCNTs.

In a present embodiment, an array of substantially identical SWCNTs is (i) coated, as in the first embodiment, or (ii) doped, as in the second embodiment, a temperature gradient is imposed on the array, and a value of an electrical parameter is determined for each coating or doping tested, and a change in sensitivity is determined or estimated as a function of the local temperature value or gradient.

In a further embodiment, a sequence of substantially identical SWCNTs are (i) coated, as in the first embodiment, or (ii) doped, as in the second embodiment, different relative humidity values (e.g., 0, 15, 30, 50, 70 and 90 percent) or other environmental parameter values are imposed on each SWCNT sensor, a value of an electrical parameter is determined for each coating or doping tested, and a change in sensitivity is determined or estimated as a function of changes in the environmental parameter value.

In another embodiment, an array of substantially identical SWCNTs is (i) coated, as in the first embodiment, or (ii) doped, as in the second embodiment. A value of an electrical parameter is determined for a selected local temperature and a selected local relative humidity, at the end of each of a sequence of selected time intervals, with temporal lengths from a few seconds to six months or more. A change in baseline and sensitivity or “drift” is determined or estimated as a function of elapsed time. An algorithm is presented that can compensate for this drift in baseline and sensitivity, as a function of time. The resultant value is used to identify and confirm the presence of disease in the provided breath sample.

In a preferred embodiment, the primary sensor contains a network of modified Single Wall Carbon Nanotubes that is connected to a controllably variable voltage difference or current source. It is used to detect the presence and/or concentration of a gas component, such as a halogen (e.g., Cl 2), hydrogen halides (e.g., HCl), a hydrocarbon (e.g., CnH2n+2), an alcohol, an aldehyde, or a ketone, to which an unmodified SWCNT is substantially non-reactive. The molecular compounds or biomarkers related to a particular physiological condition is then compared to results of the test.

In a certain embodiment, the electrical parameter values (“EPVs”), comprise electrical current, voltage difference, resistance, impedance, conductance, or capacitance. An EPV change value AEPV may be positive or negative, depending upon interaction between the specified component and the functionalized NS. Each NS in a sub-array is connected at its first end and second end to first and second electrodes, respectively, and so a AEPV measurement mechanism is also connected between the first and second electrodes.

In a certain embodiment, the parameter change value depends monotonically, not necessarily linearly, upon concentration of the gas component. Two general algorithms are presented for estimating concentration value(s), or upper or lower concentration bounds on such values, from measured differences of response values.

In the present embodiment, the first procedure of pattern recognition or discrimination is implemented by comparing magnitudes of differences of normalized AEPV values for the reference set and for the patient, summed over the different sensors for each of the specified components SCm. For each of these sums that is no greater than a threshold number, which may depend upon the specified component SCm1 the system interprets this condition as indicating that this specified component is likely present in a substantial or non-negligible concentration in the patient's sample gas (a “surviving” subset of specified components).

In yet another instance of the present embodiment, for a sum that is greater than the corresponding threshold number, the system optionally interprets this condition as indicating that this specified component is likely present, if at all, in a negligible concentration in the patients sample gas.

In a further embodiment, the specific components that survive this comparison process are then subjected to a second procedure. Calibration parameters are estimated, relating a polynomial of concentration values x for a fixed, surviving specified component to each of the reference set of AEPV values. A second sum of magnitudes of differences between the users, suitably weighted, and the calibrated AEPV values for the surviving specified components, summed over the different sensors is provided. An optimum numerical value of this non-negative “suitable weight” is expressed as a linear or quadratic polynomial in the concentration value that minimizes the second sum, and this optimum combination is used to estimate the concentration value of each of the surviving specified components in a sampled gas.

In a yet another embodiment of the present invention, the first procedure identifies specified components that are present in non-negligible concentrations in the patient's sample gas and identifies which of these estimates may be reasonably accurate. The estimated concentration values for the surviving components are used to estimate whether a specific physiological condition “condition D” may be present.

In still another embodiment, the device utilizes artificial intelligence and machine learning to compile the provided sensor data into a detailed report on all the tested parameters. An artificial intelligence algorithm is used to compare the reported parameters to a database of known combinations of biometrics and biomarkers. Unrecognized compounds or particles are mapped for future comparative analysis.

In a preferred embodiment, the results of the testing array are sent to the processor and memory systems which then render a list of recommended diet, nutritional supplement, and exercise programs that would move the user to a desired level of increased performance.

As an example: In an embodiment of the present invention, the SWCNT sensor is functionalized to detect the presence and concentration of Acetone in the breath for the purpose of measuring ketosis levels, energy expenditure, fat burn rate, and blood PH levels. As an example of actionable recommendations, the nutritional supplement Biothin® Probiotic at 250 mg/day is recommended if acetone levels are elevated. Biothin Probiomtic Promotes healthy abdominal fat storage and minimal white visceral fat. It also supports healthy, glucose sensitivity and blood glucose levels already in the normal range. Biothin Promotes healthy weight management and other factors, including body mass index as well as normal waist and hip circumference ratios. It also Promotes healthy fatty acid production and breakdown, optimal digestion and regularity.

In an embodiment of the present invention, the SWCNT sensor is functionalized to detect the presence and concentration of Carbon Monoxide in the breath for the purpose of measuring for bacterial infections and sepsis.

In an embodiment of the present invention, the SWCNT sensor is functionalized to detect the presence and concentration of Ethane in the breath for the purpose of measuring levels of lipid peroxidation, digestive health, food intolerances, airway inflammation, and oxidative stress.

In an embodiment of the present invention, the SWCNT sensor is functionalized to detect the presence and concentration of Pentane in the breath for the purpose of metabolic rate, respiratory health, stress, and muscle damage.

In an embodiment of the present invention, the SWCNT sensor is functionalized to detect the presence and concentration of Isoprene in the breath for the purpose of measuring for metabolic disorder, increased risk of disease, cholesterol biosynthesis, physical stress, and lung injury.

In an embodiment of the present invention, the SWCNT sensor is functionalized to detect the presence and concentration of Nitric Oxide in the breath for the purpose of measuring oxidative stress, inflammation, immune system health, metabolic acidosis, oxygenation levels, kidney failure, and air pollution saturation.

In an embodiment of the present invention, the SWCNT sensor is functionalized to detect the presence and concentration of Hydrogen Peroxide in the breath for the purpose of measuring respiratory health, monitoring asthma, and COPD.

In an embodiment of the present invention, the SWCNT sensor is functionalized to detect the presence and concentration of molecular Hydrogen (H2) in the breath for the purpose of measuring digestive metabolic rate, fermentation rate, cellular power generation, intestinal inflammation, and overall reaction to stress.

In an embodiment of the present invention, the SWCNT sensor is functionalized to detect the presence and concentration of IL-6 (Interluekin-6) in the breath for the purpose of measuring overall physical fitness, system-wide inflammation, infection, and cardiovascular disease.

In an embodiment of the present invention, the SWCNT sensor is functionalized to detect the presence and concentration of C-reactive protein (CRP) in the breath for the purpose of measuring inflammation, autoimmune disorder, and cardiovascular disease.

In an embodiment of the present invention, the SWCNT sensor is functionalized to detect the presence and concentration of Dopamine in the breath for the purpose of measuring overall mental wellbeing, sleep effectiveness, mental focus, and depression.

In an embodiment of the present invention, the SWCNT sensor is functionalized to detect the presence and concentration of Amyloid Beta in the breath for the purpose of measuring overall mental focus, cognitive decline, and general physical fitness level.

In another preferred embodiment, the device's software API available for developers to directly integrate the invention into their existing telemedicine systems. The on-board Bluetooth chip enables the device to integrate any Bluetooth medical device available. The 3rd party integrated Bluetooth devices will vary in quality and sensitivity based on the manufacturing techniques.

In another embodiment, the system employs a combination of heuristic artificial intelligence search methods for identifying the molecular compounds associated with the target subject. The BFS, UCS, and A* methods have been identified as appropriate for this application due to their time complexity, space complexity, Optimality, and completeness.

In another instance of the present embodiment, the BFS (Breadth First Search) algorithm has all nodes expanding level by level. It first expands all the nodes at first level in the search tree, then expands all the nodes of the second level and this way it reaches the goal. In Breadth First Search the frontier is actualized as a queue which works as First In First Out (FIFO). It is a poor strategy when all solution has a long way length or then again there is some heuristic information accessible. It is not utilized when memory requirement is high.

For example, consider the graph 1800 below (FIG. 18). Table 1 shows open list and closed list for BFS. The open list is a set of nodes yet to explore, and the closed list is a set of nodes already having been explored. At level 0 node 1 is expanded first. Children of nodes 1-2, 3, and 4 are added to the queue. Then according to First In First Out approach node 2 is expanded and child of node 2-6 is added to the queue. In this way, the search reaches the goal state. This method is used to track the geolocation and altitudinal range of the device and target subject.

In yet another instance of the embodiment, the system uses the UCS (Uniform Cost Search) to expand the node with low-cost path. It is implemented using the priority queue. To calculate cost of every node, consider this equation, c(m)=c(n)+c(n, m). where c(m) is the cost of the current node, c(n) is the cost of the previous node, and C (n, m) is the weight of the edge. The successor can be removed which are already in a queue with higher cost. Time complexity is O(b [symbol] 1+C*/e [symbol] ) and space complexity is O(b [symbol] 1+C*/e [symbol] ), where C is the optimal solution cost and each activity costs at least ε.

For example, consider graph 1900 (FIG. 19). Open list and closed list for UCS are shown in Table 1. This algorithm is used to path find comparisons between identified biomarkers and know medical conditions or virus types.

TABLE 1

| OPEN LIST | CLOSED LIST |
|---|---|
| 1 [symbol] | 1 [symbol] |
| 2⁽²⁾, 5⁽³⁾ | 5⁽¹⁾ |
| 2⁽²⁾, 9⁽²⁾ | 2⁽²⁾ |
| 9⁽²⁾, 6⁽³⁾, 3⁽³⁾ | 9⁽²⁾ |
| 6 [symbol] 3⁽³⁾, 10 [symbol] | 3⁽²⁾ |
| 6 [symbol] , 10⁽¹⁰⁾, 4 [symbol] | 6 [symbol] |
| 10 [symbol] , 4 [symbol] , 7 [symbol] , 10 [symbol] | 4⁽⁵⁾ |
| 7 [symbol] , 10⁽⁹⁾, 8 [symbol] | 7⁽⁶⁾ |
| 10⁽⁹⁾, 8 [symbol] , 11 [symbol] | 8 [symbol] |
| 10⁽⁹⁾, 11 [symbol] , 12 [symbol] | 10⁽⁹⁾ |
| 11 [symbol] , 12 [symbol] , 11 [symbol] | 11 [symbol] |
| 12 [symbol] , 12 [symbol] -Goal state | [symbol] |

[symbol]

indicates data missing or illegible when filed

In yet another instance of the present embodiment, the system uses the [symbol] artificial intelligence algorithm. [symbol] algorithm joins highlight of Uniform Cost Search and pure heuristic search to productively calculate optimal solution. For computing cost of every node, consider this equation, f(m)=c(m)+h(m), where c(m)'c(n)+c(n, m), h(m) is heuristic function. f(m) computes the most reduced aggregate cost. Most reduced value of f is chosen at each node for expansion. Euclidean distance (used when allowed to move in any direction), Manhattan distance (used when allowed to move in only four directions-right, left, top, and bottom) or Diagonal distance (used when allowed to move in eight directions, same as the movement of king in chess) are used as Heuristic function.

If the value of f of two nodes is the same, then the node having lowest h value is chosen for expansion. The calculation ends when an objective is decided for expansion. Admissible heuristic function (h(n)≤ [symbol] (n)) brings visited node back from the closed list to open list to get an optimal solution. Time complexity is O(bd) and space complexity is O(bd), where b is branching factor and d is solution depth. For example, consider graph 2000 (FIG. 20). Open and closed list associated with the example is shown in table 2.

TABLE 2

| OPEN LIST | CLOSED LIST |
|---|---|
| 1 [symbol] | |
| 2 [symbol] , 5 [symbol] | |
| 5 [symbol] , 3 [symbol] , 6 [symbol] | |
| 5 [symbol] , 3 [symbol] | |
| 3 [symbol] , 7 [symbol] , 10 [symbol] , 9 [symbol] | |
| 3 [symbol] , 7 [symbol] , 9 [symbol] , 11 [symbol] | |
| 3 [symbol] , 7 [symbol] , 9 [symbol] , 12 [symbol] -Goal | |
| state | |

[symbol]

indicates data missing or illegible when filed

In another instance of the present embodiment, the system uses steps to reach the goal state from source.
1. Start from the source node, add it open list.
2. Explore all the nodes which are adjacent to the node which is in open list.
3. Calculate the cost for all the nodes discovered in step 2 and place them in open list in increasing order based on cost.
4. Move current working node, from the open list to closed list.
5. The first node in open list will become the current working node.
6. Repeat step 2 to 5, if the current working node is not goal state.
7. The closed list gives the shortest path and the value of last cost function obtained gives the optimal cost.

In yet another instance of the present embodiment, the system primarily uses the Deep belief network (DBN) machine learning algorithm for VOC discrimination. DBN is a generative graphical model, or alternatively a class of deep neural network, composed of multiple layers of latent variables (“hidden units”), with connections between the layers but not between units within each layer.

When trained on a set of examples without supervision, a DBN can learn to probabilistically reconstruct its inputs. The layers then act as feature detectors. After this learning step, a DBN can be further trained with supervision to perform classification.

DBNs can be viewed as a composition of simple, unsupervised networks such as Restricted Boltzmann machines (RBMs) or autoencoders, where each sub-network's hidden layer serves as the visible layer for the next. An RBM is an undirected, generative energy-based model with a “visible” input layer and a hidden layer and connections between but not within layers. This composition leads to a fast, layer-by-layer unsupervised training procedure, where contrastive divergence is applied to each sub-network in turn, starting from the “lowest” pair of layers (the lowest visible layer is a training set).

The training method for the RBM's is the Contrastive Convergence method (CD). CD provides an approximation to the maximum likelihood method that would ideally be applied for learning the weights. In training a single RBM, weight updates are performed with gradient descent via the following equation:

$$\text{?}$$

$$\text{?} \text{indicates text missing or illegible when filed}$$

where, p(v) s the probability of a visible vector, which is given by

E(v, h)

This is the partition function used for normalizing and is the energy function assigned to the state of the network. A lower energy indicates the network is in a more “desirable” configuration. The gradien

$$\text{?}$$

$$\text{?} \text{indicates text missing or illegible when filed}$$

has the simple form [symbol] vᵢhⱼ [symbol] _{data}− [symbol] vᵢhⱼ [symbol] _{model} where [symbol] . . . [symbol] ₚ represent averages with respect to distribution p. T he issue arises in sampling because this requires extended alternating Gibbs sampling. CD replaces this step by running alternating Gibbs sampling for n steps (values of n=1 perform well). After n steps, the data is sampled, and that sample is used in place of [symbol] vᵢhⱼ [symbol] _{model}.

The CD procedure works as follows:
1. Initialize the visible units to a training vector
2. Update the hidden units in parallel given the visible units

$$\text{?}$$

$$\text{?} \text{indicates text missing or illegible when filed}$$

is the sigmoid function and bⱼ is the bias of hⱼ.
3. Update the visible units in parallel given the hidden units

$$\text{?}$$

$$\text{?} \text{indicates text missing or illegible when filed}$$

is the bias of vᵢ. This is called the “reconstruction” step.
4. Re-update the hidden units in parallel given the reconstructed visible units using the same equation as in step 2.
5. Perform the weight update: Δwᵢⱼ∝ [symbol] vᵢhⱼ [symbol] _{data}− [symbol] vᵢhⱼ [symbol] _{reconstruction}.

Once an RBM is trained, another RBM is “stacked” atop it, taking its input from the final trained layer. The new visible layer is initialized to a training vector, and values for the units in the already-trained layers are assigned using the current weights and biases. The new RBM is then trained with the procedure above. This whole process is repeated until the desired stopping criterion is met. Although the approximation of CD to maximum likelihood is crude (does not follow the gradient of any function), it is empirically effective.

In a further instance of the present embodiment, the system uses the BPS (Greedy Search) algorithm. Best First Search is a merger of Breadth First Search and Depth First Search. Best First Search is implemented using the priority queue. The advantage of Depth First Search is that it gives a solution without calculating all node. Breadth First Search arrives at a solution without search guaranteed that the procedure does not get caught. Best First Search, being a mixer of these two, licenses exchanging between paths. At each stage the nodes among the created ones, the best appropriate node is chosen for facilitating expansion, might be this node have a place to a similar level or different, hence can flip between Depth First and Breadth First Search. It is also known as greedy search. Time complexity is 0(bd) and space complexity is 0(bd), where b is branching factor and d is solution depth.

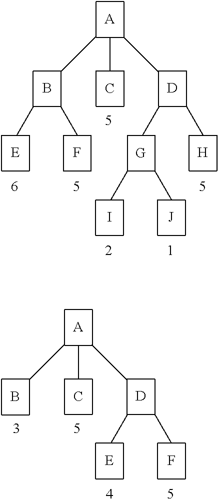

$$p ( v ) = \frac{1}{Z} {\sum }_{h} {e}^{- E ( v , h )}$$

In a certain embodiment, a Convolutional Neural Network (CNN), is programmatically made operable to analyze the SWCNT breath sensor data and extract relevant features related to discreet molecules in a user provided breath sample.

In an instance of a preferred embodiment, a Recurrent Neural Network (RNN), is programmatically made operable to analyze integrated biometric sensor data and capture the temporal dynamics of physiological signals from the integrated sensors.

In yet another instance of the present embodiment, a Gradient Boosting Tree (GBT), is made operable to analyze third-party fitness app data and identify patterns related to exercise intensity, duration, and type.

In a further instance of the present embodiment, a Bayesian Network (BN), is made operable to integrate the outputs of the above models and create a comprehensive assessment of the athletic performance and recovery of a human subject.

In a preferred embodiment, the target subject initiates a testing sequence by tapping on one of the EKG sensors pads or selecting “begin test” from the device control application. The subject then blows into the puffer nozzle for 30 seconds. The breath particles are then deposited on the SWCNT sensor array. The sensor array instantly detects a preset of target biomarkers and returns the query with a matching physical condition. This sensor also detects athletic readiness, metabolic function, and metal alertness. The result of the puffer test is then sent to the main processor for sensor data compilation.

In this same embodiment, the gases breathed into the puffing chamber are exhausted out of the bottom of the device through the puffer vent. The particles deposited on the SWCNT sensor array are exposed to UV light via the UV LED sterilization array. The UV light destroys the deposited particles after 100 seconds and a heating element heats the swcnt senor for 30 seconds. A micro fan located on the SWCNT board assists in removing the destroyed particles. The exhausted air passes over a MEMS air flow sensor while the subject is blowing and measures lung volume, capacity, rates of flow and gas exchange.

In yet another instance of this embodiment, the subject places two fingers from each hand on the EKG sensor pad array for thirty seconds. Upon completion, the LED indicator will flash, indicating the subject should move on to the next test. The EKG reading will provide data regarding the hear health of the subject.

In yet another instance of this embodiment, the subject holds his/her right pointer finger over the Pulse oximeter and body temp sensor. After holding for 15 seconds, the subject removes their finger. The pulse oximeter and temperature readings are then sent to the main processor for sensor compilation.

In yet another instance of the present embodiment, the subject plugs in a three lead EEG electrode sensor into the 3.5 mm EEG sensor port. The subject then applies the disposable EEG sensors to their foreheads. Once the sensors are plugged in, the reading starts immediately. The EEG reading takes 20 minutes and then provides the main processor with a return report.

In yet another instance of the present embodiment, the device takes real-time samples of air quality, ambient room temperature, and humidity level. The sum of the sampled data is compiled into a close-range environmental conditions report. This data is added to geolocation data such as weather, outdoor relative humidity and, barometric pressure, and light intensity. This data compilation is used to image a detailed environmental conditions report.

The claimed invention is:

1. A system, device, and method for athletic readiness and recovery profiling, comprising:
a mouthpiece connected to a housing, the mouthpiece operable to receive the exhaled breath of a human subject;
a chemiresistor sensor module disposed in the breath sampling chamber of the housing,
the sensor module operable to detect one or more volatile organic compounds (VOCs) associated with athletic readiness and recovery in the exhaled breath of a human subject, and further operable to collect data associated with the detection of the one or more VOCs,
a chemiresistor sensor module wherein the sensor module comprises of at least one pair of interdigitated electrodes fabricated by embedding the electrodes on a printed circuit board (PCB) substrate with a finger gap size of 200 μm and depositing chemically functionalized carbon nanotubes between the electrodes;
a plurality of integrated biometric sensors including heart rate, blood pressure, EEG bio-electrical impedance analysis, ECG, airflow, temperature, and humidity the sensors operable to collect biometric data from the human subject;
a first communication interface arranged to receive a plurality of gas measurements generated by at least one chemiresistive gas sensor of a breath analysis device;
a microprocessor to gather and analyze data from the connected sensors that is attached to a memory module configured to store the collected sensor data;
a communication module disposed in the housing and in communication with the microprocessor, memory module, sensor module, the integrated biometric sensors, operable to transmit collected data from the sensor modules to a mobile communication device associated with the human subject; and
a user interface screen to display the compiled sensor data.

2. The system of claim 1, wherein the communication device is further operable to gather biometric sensor data from third party health and fitness devices by means of application programming interface queries.

3. The system of claim 1, wherein the communication module is further operable to send the combined SWCNT sensor and integrated biometric sensor data to a remote server to be processed in real-time using artificial intelligence models including, but not limited to:
a Convolutional Neural Network (CNN), operable to analyze the SWCNT breath sensor data and extract relevant features related to discreet molecules in a gas sample;
a Recurrent Neural Network (RNN), operable to analyze integrated biometric sensor data and capture the temporal dynamics of physiological signals from the integrated sensors;
a Gradient Boosting Tree (GBT), operable to analyze third-party fitness app data and identify patterns related to exercise intensity, duration , and type; and
a Bayesian Network (BN), operable to integrate the outputs of the above models and create a comprehensive assessment of the athletic performance and recovery of a human subject.

4. The system of claim 1, wherein the VOC's comprise one or more of acetone, ethane, pentane, isoprene, nitric oxide, hydrogen peroxide, carbon dioxide, hydrogen, inteluekin-6, dopamine, amyloid beta, water, lactic acid, acetaldehyde, ammonia, hydrogen sulfide, ferritin, hexane, c-reactive protein, and certain amino acids.

5. The system of claim 1, wherein the micro-processor and memory are configured to perform the following steps:
receiving a sequence of electrical parameter values measured from each nanostructure sensor of the plurality of nano sensors, each of the sequences corresponding to measured electrical values from a measurement mechanism;
generating a normalized amplitude value for one of the measured electrical values measured from each of the plurality of nanostructure sensors to form a set of amplitude values for the sample gas;
determining the presence of at least a first specified component in the sample gas by:
comparing a normalized amplitude value for the first nanostructure sensor for the sample gas with a reference amplitude value for the first nanostructure sensor for the first specified component to generate a compared value for the first nanostructure sensor;
repeating the comparing step for each of the other sensors of the plurality of nanostructure sensors to generate a set of the compared values;
aggregating the compared values to generate a set of compared values,
wherein the aggregating includes a weighted summation of the compared values, and based on the aggregated compared values, determining whether the specified component is likely present in the sample gas.

6. The system of claim 1, wherein at least the first SWCNT sensor and the second SWCNT sensor of the sensor array are functionalized with different reactive chemicals to create a differential in selectivity and sensitivity to a specified gas component.

7. The system of claim 1, wherein each of the SWCNT sensors in the sensor array are differently sensitive to at least two of the specified component gases.

8. The system of claim 1, further comprising a measurement mechanism electrically coupled to each of the individual SWCNT nanostructures operable for the measuring the electrical parameter values generated by each nanostructure sensor in response to exposure to the sample gas.

9. The system of claim 1, wherein the electrical parameter values include one or more of electrical current voltage difference, resistance, impedance, conductance and capacitance.

10. The system of claim 1, wherein the sensor array contains at least two and as many as 128 SWCNT nanostructure sensors.

11. The system of claim 1, wherein the plurality of SWCNT sensors is refreshed for repeated testing after being exposed to ultraviolet lights from light-emitting diodes and a heating element for a duration of 1 to 100 seconds.

12. The system of claim 1, wherein the step of determining whether the specified component is likely present in the sample gas includes the steps of
generate an error value based on the aggregated compared values;
comparing the error value with a threshold error value; and
determine presence of the sampled gas if the error value is less than the threshold error value.

13. The system in claim 1, wherein the sample gas is received from the user by inhaling a deep breath and exhaling the contents of the lung completely in one continuous breath;
wherein the typical exhalation lasting between 4-6 seconds and a sample will contain approximately 5 breath samples over a 30 second period;
wherein the highest concentration of VOC's is found in the aveolar air that is exhaled at the very end of the sample.

14. The system of claim 1, the micro-processor and memory systems further configured to perform the steps of:
analyzing a reference sample gas, the reference gas comprising a mixture of healthy or optimal sample gas having a known concentration of a specified component, the analyzing comprising determination of two or more electrical parameter values that associate the known concentration with a measured electrical value for the reference sample gas; and
determining a concentration of the specified component in the sample gas based on:
the set of measured electrical values for the sampled gas, and
the two or more parameter values as determined in the analyzing the reference sample gas.

15. The system of claim 14, wherein the process of identifying two or more parameter values that link the measured electrical value with the known concentration involves determining either a linear or quadratic relationship between the known concentration and the measured electrical value.

16. The system of claim 14, the processor and memory system is further configured to perform the steps of:
determining the athletic fitness level of a user based upon specified gas components as compared to the relative electrical values of gas samples provided by elite athletes that have completed similar testing as part of the comparison matrix;
determining the athletic fitness level of a user based on a data compilation of biometric sensor readings over a period of time as compared to a database of similar biometric readings from elite athletes as part of the comparison matrix; and
determining the fitness level of a user by gathering fitness data from third party fitness tracking devices to validate or invalidate compiled data from integrated sensors disposed in the housing of the presented invention.

17. A system, device, and method for athletic readiness and recovery profiling substantially as shown and described herein.
